(19) United States
(12) Patent Application Publication
(10) Pub. No.: US 2013/0210784 A1
(43) Pub. Date: Aug. 15, 2013

(54) COMBINATIONS AND METHODS FOR TREATING NON AGE-RELATED HEARING IMPAIRMENT IN A SUBJECT

(71) Applicant: The Washington University, (US)

(72) Inventors: Jianxin Bao, St. Louis, MO (US); Richard Chole, St. Louis, MO (US)

(73) Assignee: THE WASHINGTON UNIVERSITY, St. Louis, MO (US)

(21) Appl. No.: 13/764,352

(22) Filed: Feb. 11, 2013

Related U.S. Application Data
(60) Provisional application No. 61/597,411, filed on Feb. 10, 2012.

(51) Int. Cl. A61K 31/57; A61K 31/421; A61K 31/423; A61K 31/4015

(52) U.S. Cl. CPC A61K 31/57; A61K 31/4015; A61K 31/421; A61K 31/423
USPC 514/171

(57) ABSTRACT

The present invention generally relates to treating non age-related hearing impairments. More specifically, the present invention provides combinations and methods for treatment and prevention of non age-related hearing impairment in a subject.

### GOVERNMENTAL RIGHTS

This invention was made with government support under grant no. R21DC010489 and DC011793 awarded by the National Institute of Deafness and Other Communication Disorders. The government has certain rights in the invention.

### FIELD OF THE INVENTION

The present invention generally relates to treating non age-related hearing impairments. More specifically, the present invention provides combinations and methods for treatment and prevention of non age-related hearing impairment in a subject.

### BACKGROUND OF THE INVENTION

In the United States, almost thirty million people suffer from some degree of hearing loss or deafness and the condition costs the nation more than 50 billion dollars each year, more than epilepsy, multiple sclerosis, spinal injury, stroke, Huntington's, and Parkinson's disease combined. There are three types of hearing loss, namely conductive hearing loss, sensorineural hearing loss, and mixed hearing loss, a combination of conductive and sensorineural hearing loss. Conductive hearing loss results from impairment of the external or middle ear, which is commonly mechanical in nature, i.e., impacted earwax, presence of a foreign body, ear infection (otitis media, external otitis), and thus can be corrected by medicine and/or surgery. Sensorineural hearing loss includes sensory hearing loss, which is due to disorders in the cochlea, and neural hearing loss, which results from damage to or the absence of the vestibulocochlear nerve, also referred to as cranial nerve VIII or the auditory nerve. The vast majority of cases of hearing loss are sensorineural and are caused by a loss of hair cells in the cochlea.

Noise-induced hearing loss is the second most common form of sensorineural hearing loss (after age-related hearing loss). Noise-induced hearing loss is the loss of hearing resulting from exposure to loud noises. Both acute and chronic exposure to loud noises can cause hearing loss, but it is more common and more pronounced for subjects to experience significant hearing loss due to chronic exposure to loud noises. Many workers, particularly in manufacturing, are exposed to such loud noises at the workplace, making noise the most common occupational hazard. Other non age-related hearing loss may be caused, for example, by surgical procedures, toxins, and various pathological conditions.

Development of an efficacious treatment for non age-related hearing loss has been hampered by the complex array of cellular and molecular pathways involved in hearing loss. Steroids have been shown to have some effect on hearing loss; however, high doses of steroids may have undesired systemic effects. Currently, there are no effective pharmacological agents are approved by the FDA to diminish or prevent permanent hearing loss. Therefore, there is a need in the art for therapeutics capable of treating or preventing non age-related hearing loss.

### SUMMARY OF THE INVENTION

The present invention relates to compositions and methods for treatment and prevention of non age-related hearing impairments.

In one aspect, the disclosure provides a combination, the combination comprises a corticosteroid and an antiepileptic drug in an amount therapeutically effective to treat or prevent non age-related hearing impairments in a subject. The therapeutically effective amount of the corticosteroid comprises a dose ranging from about 1 to about 50 mg/kg of the body weight of the subject, and the therapeutically effective amount of the antiepileptic comprises a dose ranging from about 20 to about 350 mg/kg of the body weight of the subject.

In another aspect, the disclosure provides a method for treating or preventing a non age-related hearing impairment in a subject in need of such treatment. The method comprises administering a combination comprising a corticosteroid and an antiepileptic drug to a subject in an amount therapeutically effective to treat or prevent a non age-related hearing impairment in the subject. The therapeutically effective amount of the corticosteroid comprises a dose ranging from about 1 to about 50 mg/kg of the body weight of the subject, and the therapeutically effective amount of the antiepileptic comprises a dose ranging from about 20 to about 350 mg/kg of the body weight of the subject.

Other features and iterations of the disclosure are described in more detail herein.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

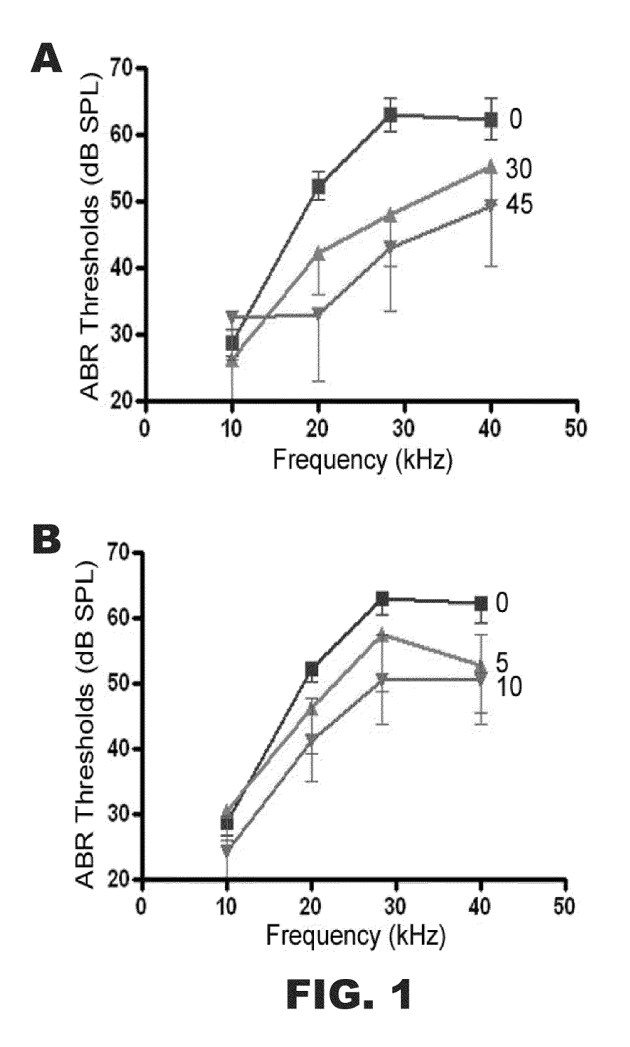

FIG. 1 depicts two plots showing prevention of hearing loss by corticosteroid drugs. (A) shows ABR (Auditory Brainstem Response) thresholds for methylprednisolone at two different doses compared to control (0, 30, and 45 mg/kg) (n=8 for each group). (B) shows ABR thresholds for dexamethasone at two different doses compared to control (0, 5, and 10 mg/kg) (n=8 for each group).

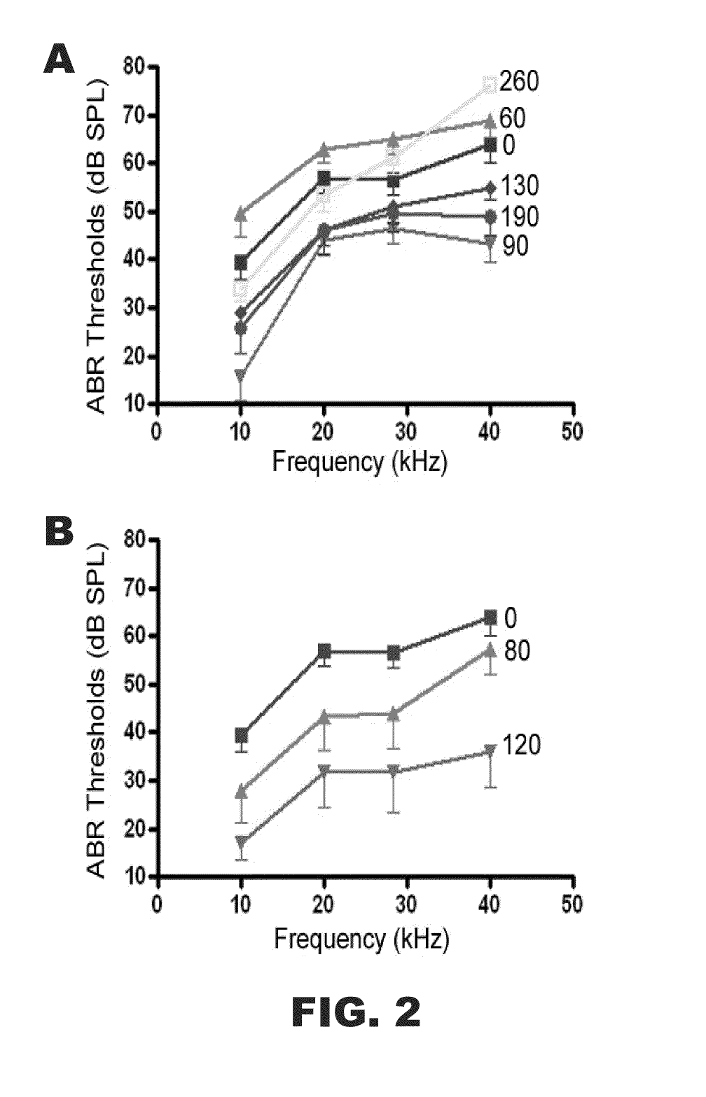

FIG. 2 depicts two plots showing prevention of hearing loss by antiepileptic drugs. (A) shows ABR thresholds for among control and various doses of ethosuximide (0 mg/kg, 60 mg/kg, 90 mg/kg, 130 mg/kg, 190 mg/kg, 260 mg/kg) (n=8 for each group). (B) shows ABR thresholds among control and various dosages of zonisamide (0 mg/kg, 80 mg/kg, 120 mg/kg) (n=8 for each group).

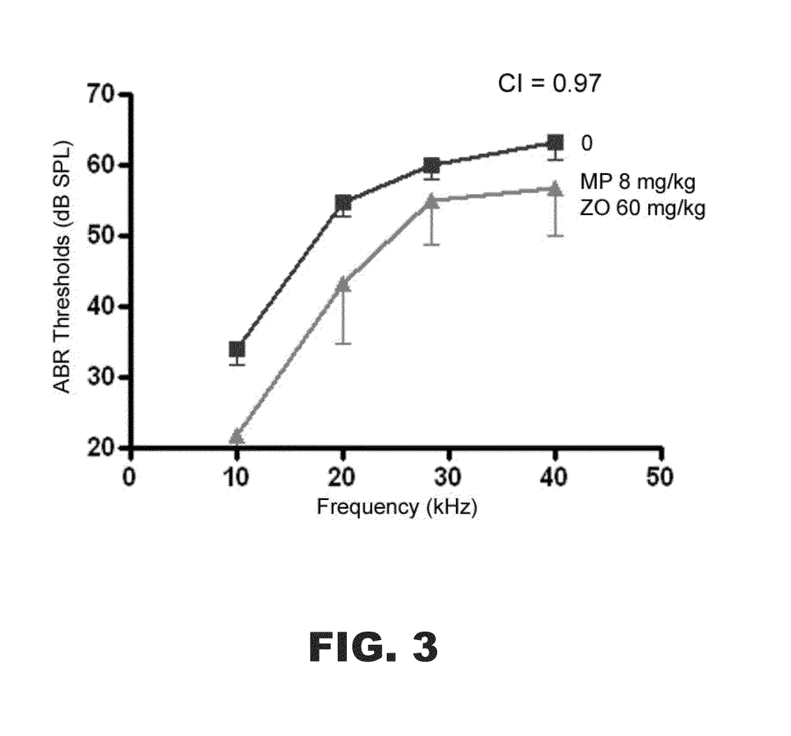

FIG. 3 depicts a plot showing prevention of hearing loss with a combination of methylprednisolone (MP) and zonisamide (ZO) at low doses (MP=8 mg/kg, ZO=60 mg/kg) for a control and a treated group (n=6 for each group). A synergistic effect was found due to the fact that CI<1.

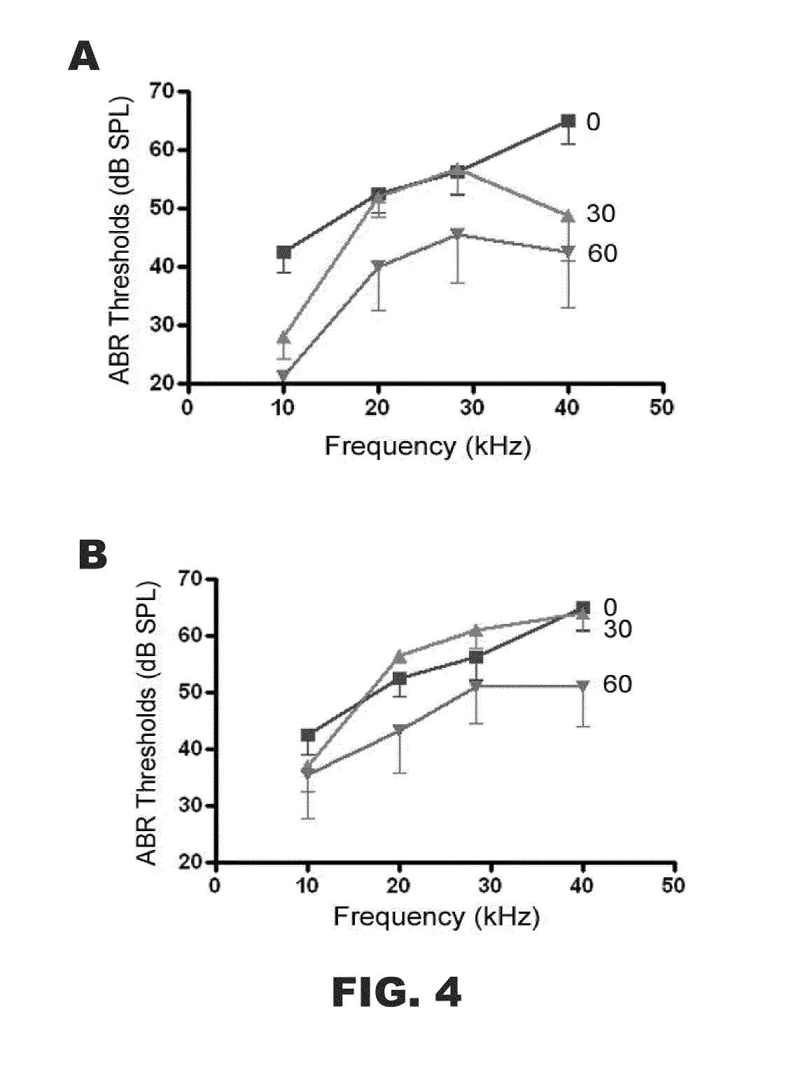

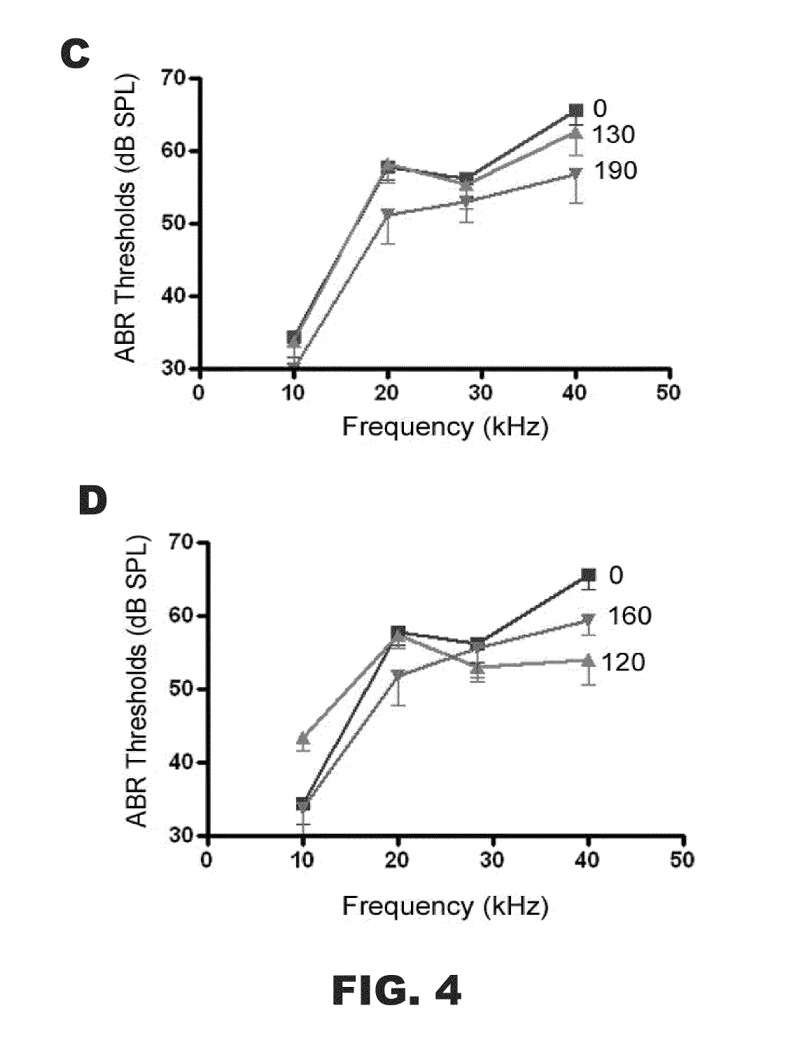

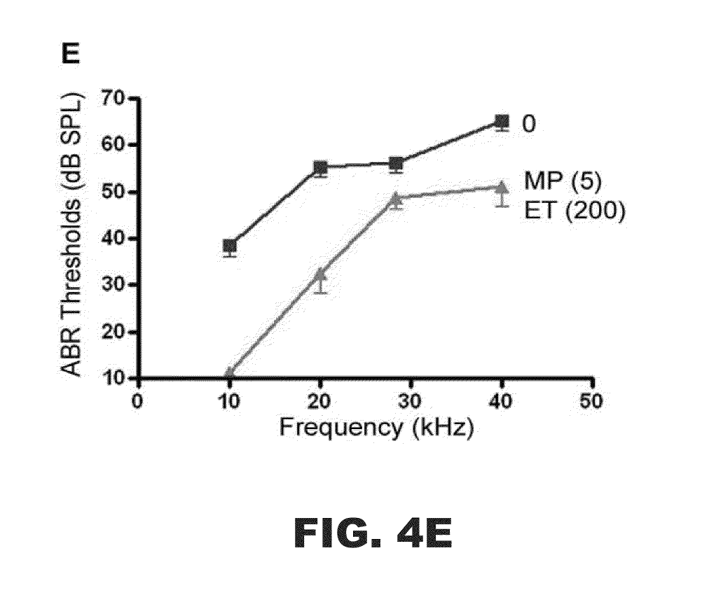

FIG. 4 depicts five plots showing treatment of hearing loss by different drug families and a combination of drug families. (A) shows ABR thresholds among control and different dosages of methylprednisolone (0, 30, and 60 mg/kg) (n=8 for each group). (B) shows ABR thresholds among control and different dosages of dexamethasone (0, 30, and 60 mg/kg) (n=9 for each group). (C) shows ABR thresholds among control and different dosages of ethosuximide (0, 130, and 190 mg/kg) (n=8 for each group). (D) shows ABR thresholds among control and different dosages of zonisamide (0, 120, and 160 mg/kg) (n=8 for each group). (E) shows ABR thresholds between control and a combination of methylprednisolone (MP) and ethosuximide (ET) (n=4 for each group). Non-control animals were given the combination in drinking water 24 hours after exposure to the noise.

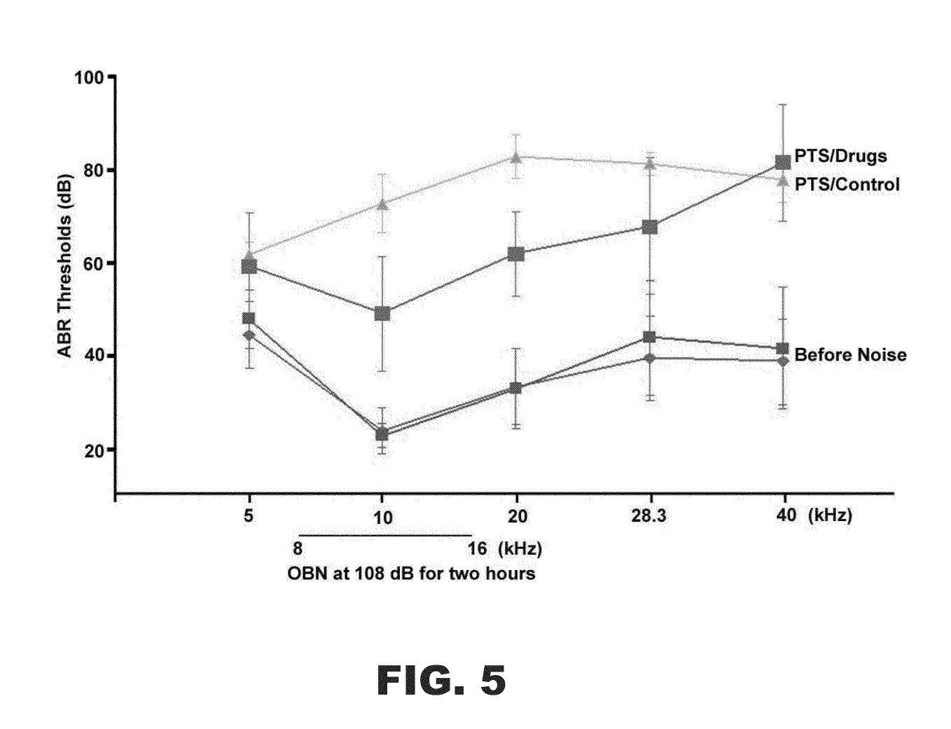

FIG. 5 depicts a plot showing ABR thresholds among control and drug treated mice. The drug treated mice (2 month-old B6.CAST mice) were given methylprednisolone (5 mg/kg) and trimethadione (200 mg/kg) given 24 hours after exposure to the 8-16 kHz OBN at 108 dB SPL for 2 hours.

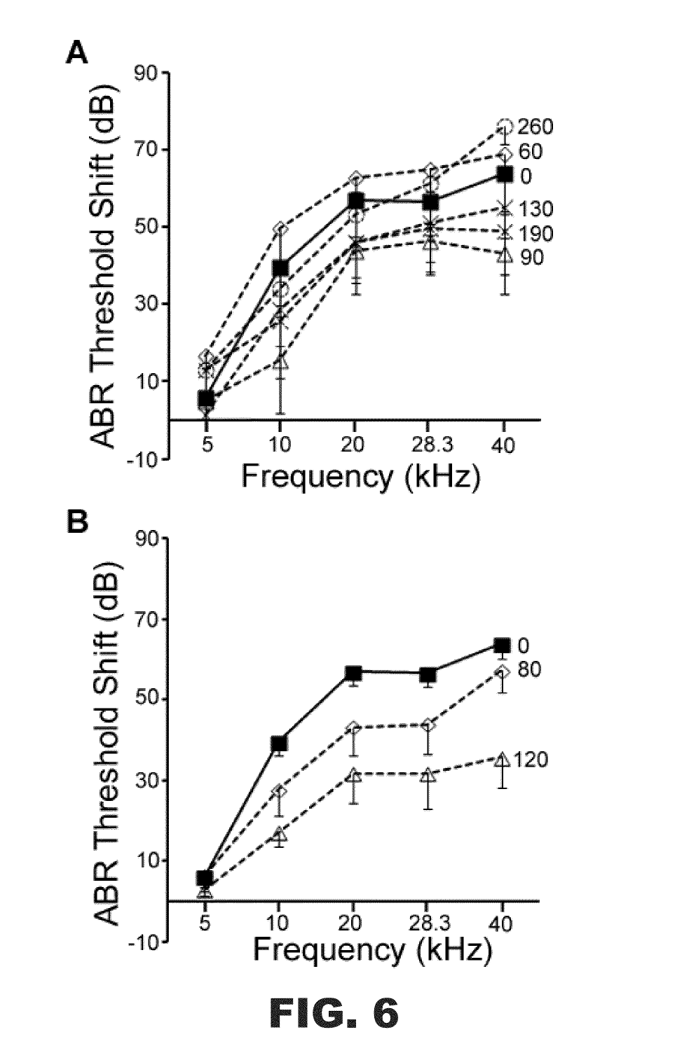

FIG. 6 depicts two plots showing prophylactic functions of ethosuximide or zonisamide. (A) ABR threshold shifts (Mean±S.D) for C57BL/J mice treated with ethosuximide two hours before noise exposure (n=8 for noise alone or noise+drug at each dosage, four mice per gender in each group). (B) ABR threshold shifts for C57BL/6J mice treated zonisamide two hours before noise exposure (n=8 for each group, four mice per gender in each group). All animals were exposed to the noise at two months old.

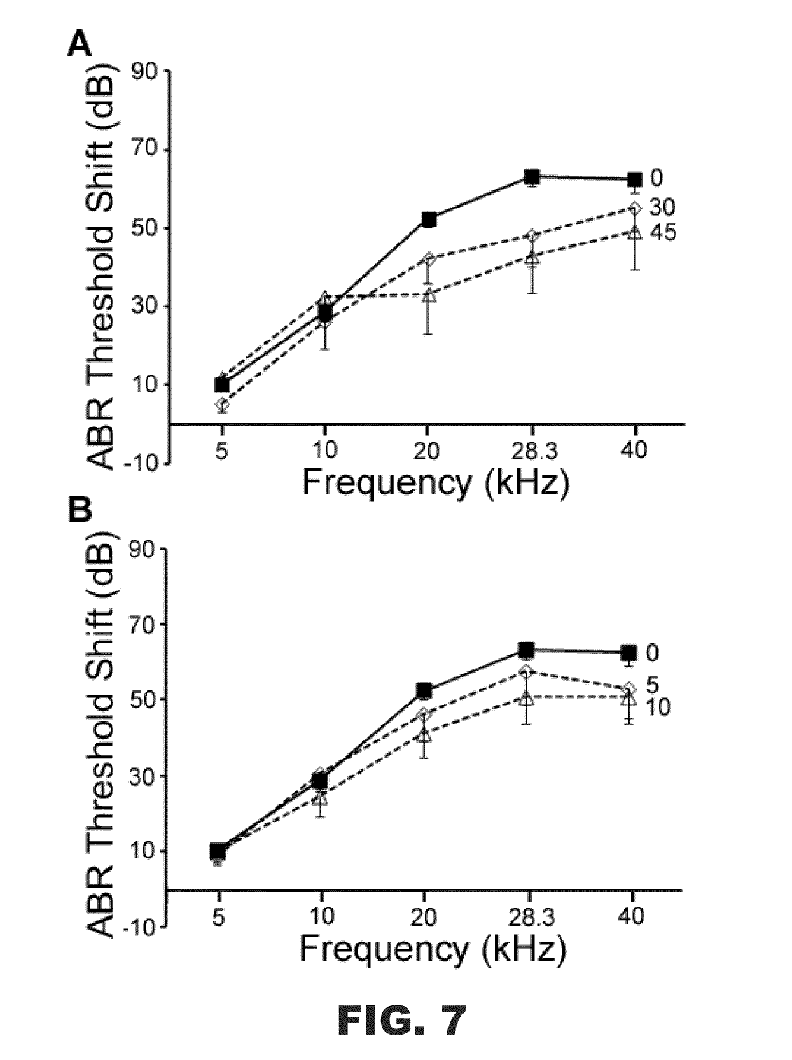

FIG. 7 depicts two plots showing prophylactic function of methylprednisolone or dexamethasone. (A) ABR threshold shifts (Mean±S.D) for C57BL/J mice treated with methylprednisolone two hour before the noise exposure (n=8 for noise alone or noise+drug at each dosage, four mice per gender in each group). (B) ABR threshold shifts for C57BL/J mice treated dexamethasone two hours before the noise exposure (n=8 for each group, four mice per gender in each group). All animals were exposed to the noise at two months old.

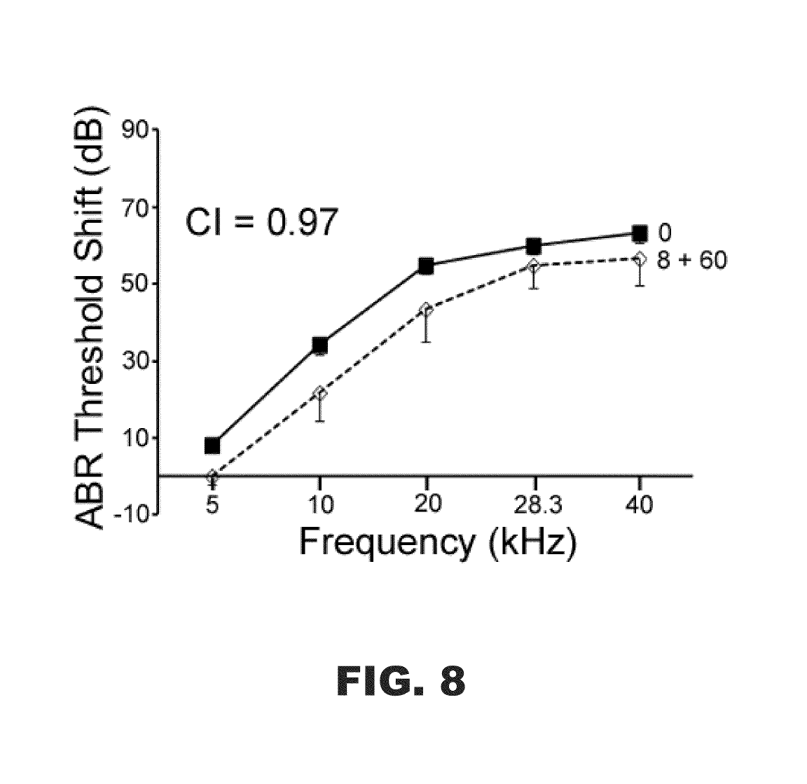

FIG. 8 depicts a plot showing synergistic function of methylprednisolone and zonisamide. ABR threshold shifts (Mean±S.D) for control C57BL/J mice (n=16, eight mice per gender) and mice treated with both methylprednisolone and zonisamide two hours before the noise exposure (n=6, three per gender). All animals were exposed to the noise at two months old.

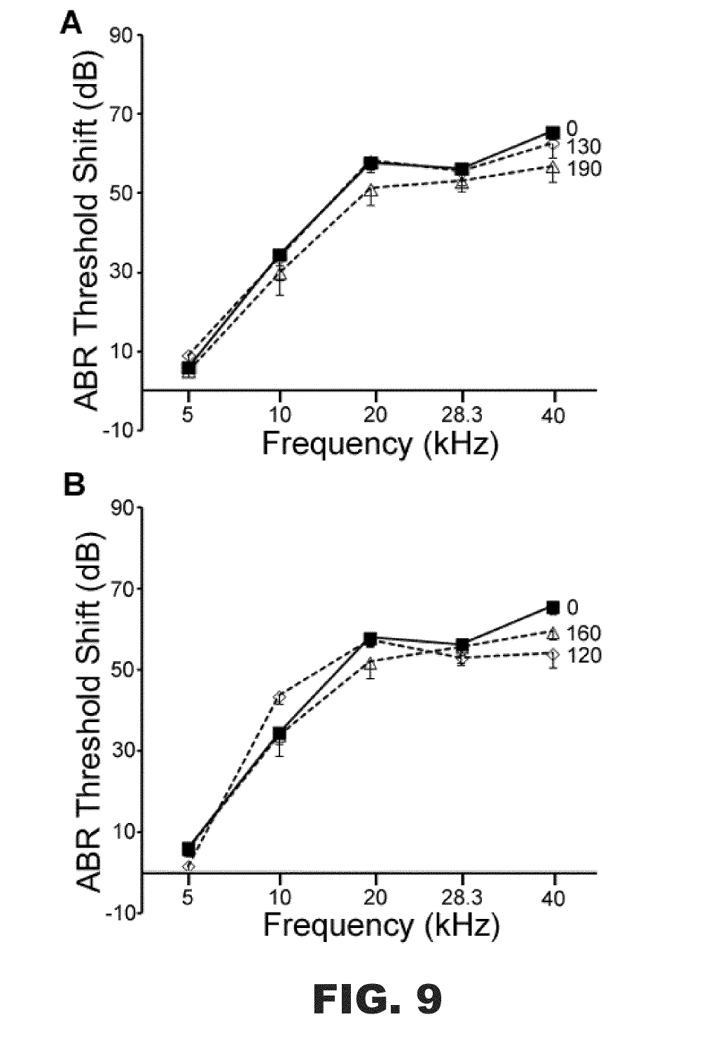

FIG. 9 depicts two plots showing therapeutic function of ethosuximide or zonisamide. (A) ABR threshold shifts (Mean±S.D) for C57BL/J mice treated with ethosuximide 24 hours after the noise exposure (n=8 for noise alone or noise+drug at each dosage, four mice per gender in each group). (B) ABR threshold shifts for C57BL/J mice treated zonisamide 24 hours after the noise exposure (n=8 for the control group and the group treated with zonisamide at 160 mg/kg, n=10 for the group treated with zonisamide at 120 mg/kg, equal gender in each group). All animals were exposed to the noise at two months old.

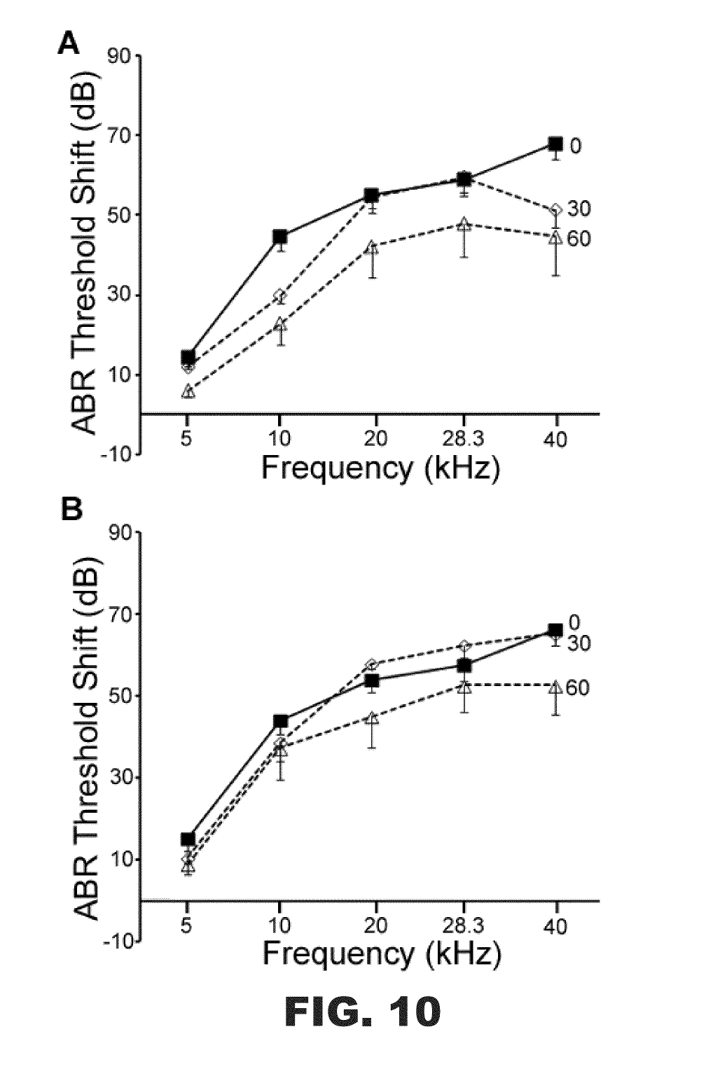

FIG. 10 depicts two plots showing therapeutic function of methylprednisolone or dexamethasone. (A) ABR threshold shifts (Mean±S.D) for C57BL/J mice treated with methylprednisolone 24 hours (n=8 for noise alone or noise+drug at each dosage, four mice per gender in each group). (B) ABR threshold shifts for C57BL/J mice treated dexamethasone 24 hours after the noise exposure (n=8 for the control group, n=10 for the group treated with dexamethasone at 30 mg/kg, equal gender in each group; n=9 for the group treated at 60 mg/kg, two males and two females as control mice, two males and three females treated with dexamethasone). All animals were exposed to the noise at two months old.

### DETAILED DESCRIPTION OF THE INVENTION

The present invention encompasses a preventative or therapeutic treatment for non age-related hearing impairments comprising a combination of therapeutics, namely a corticosteroid and an antiepileptic drug. Advantageously, such a combination has been discovered to have synergistic preventative or therapeutic activity.

The invention further provides methods for the treatment and prevention of non age-related hearing impairments by administration of a combination of the invention. The combinations may reduce permanent and temporary hearing threshold shifts in subjects experiencing non age-related hearing loss or in subjects at risk for non age-related hearing loss through various conditions including pathological conditions or exposure to risk factors such as noise, surgical procedures, toxins, and other stressors.

### I. Therapeutic Combinations

In one aspect, the invention provides a combination comprising a corticosteroid and an antiepileptic drug in an amount therapeutically effective to treat or prevent non age-related hearing impairments. In some embodiments, the combinations are formulated for pharmaceutical uses.

### (a) Corticosteroid

A combination of the invention comprises a corticosteroid. The corticosteroid is preferably a glucocorticoid which causes up-regulation of the glucocorticoid signaling pathways via interaction with the glucocorticoid receptor. Corticosteroids may be selected from hydrocortisone, cortisone acetate, prednisone, prednisolone, methylprednisolone, dexamethasone, triamcinolone, beclomethasone, fludrocortisone, deoxycorticosterone, aldosterone, and salts, enantiomers, and derivatives thereof. In a preferred embodiment, the corticosteroid is methylprednisolone.

### (b) Antiepileptic Drugs

A combination of the invention also comprises an antiepileptic. Antiepileptics include oxazolidinediones (such as paramethadione, trimethadione, ethadione), sulfonamides (such as acetazolamide, sultiame, methazolamide, and zonisamide), succinimides (such as ethosuximide, phensuximide, and mesuximide) and derivatives thereof. The anti-epileptic drug may act to block T-type calcium channels. T-type calcium channels are a voltage gated calcium channel that have low activation ranges and are characterized by their transient kinetics of inactivation.

Preferred antiepileptics include ethosuximide, trimethadione, and zonisamide.

### (c) Combinations

It has been discovered that combinations of a therapeutically effective amount of a corticosteroid and an antiepileptic are suitable for treatment or prevention of non age-related hearing impairments. The combinations comprise one corticosteroid and one antiepileptic. In alternative embodiments, the combinations comprise one or more corticosteroids and one or more antiepileptics. One preferred combination comprises methylprednisolone and ethosuximide. Another preferred combination comprises methylprednisolone and trimethadione. Still another preferred combination comprises methylprednisolone and zonisamide.

The corticosteroid may be present in the combination in an amount ranging from about 1 to about 50 mg/kg of the body weight of the subject. In some embodiments, the corticosteroid is present in the combination in an amount ranging from about 3 to about 20 mg/kg. In still other embodiments, the corticosteroid is present in the combination in an amount ranging from about 1 to 2 mg/kg, 1.5 to 2.5 mg/kg, 2 to 3 mg/kg, 2.5 to 3.5 mg/kg, 3 to 4 mg/kg, 3.5 to 4.5 mg/kg, 4 to 5 mg/kg, 4.5 to 5.5 mg/kg, 5 to 6 mg/kg, 5.5 to 6.5 mg/kg, 6 to 7 mg/kg, 6.5 to 7.5 mg/kg, 7 to 8 mg/kg, 7.5 to 8.5 mg/kg, 8 to 9 mg/kg, 8.5 to 9.5 mg/kg, or 9 to 10 mg/kg. In one preferred embodiment, the corticosteroid is present in the combination in an amount of about 8 mg/kg. In another preferred embodiment, the corticosteroid is present in the combination in an amount of about 5 mg/kg.

The antiepileptic may be present in the combination in an amount ranging from about 20 to about 350 mg/kg in different embodiments. In one embodiment, antiepileptic is present in the combination in an amount ranging from about 150 to about 250 mg/kg, in still another embodiment, the antiepileptic is present in an amount ranging from about 40 to about 80 mg/kg. In some embodiments the antiepileptic is present in an amount ranging from about 40 to 50 mg/kg, 50 to 60 mg/kg, 60 to 70 mg/kg, 70 to 80 mg/kg, 80 to 90 mg/kg, 90 to 100 mg/kg, 100 to 110 mg/kg, 110 to 120 mg/kg, 120 to 130 mg/kg, 130 to 140 mg/kg, 140 to 150 mg/kg, 150 to 160 mg/kg, 160 to 170 mg/kg; 170 to 180 mg/kg, 180 to 190 mg/kg, 190 to 200 mg/kg, 200 to 210 mg/kg, 210 to 220 mg/kg, 220 to 230 mg/kg, 230 to 240 mg/kg, 240 to 250 mg/kg, 250 to 260 mg/kg, 260 to 270 mg/kg, 270 to 280 mg/kg, 280 to 290 mg/kg, 290 to 300 mg/kg, and including ranges between and including the listed values.

In one embodiment, the corticosteroid is methylprednisolone which is present in an amount ranging from about 3 to about 8 mg/kg and the antiepileptic is trimethadione and is present in an amount ranging from about 180 to about 220 mg/kg. In one preferred embodiment, the corticosteroid is methylprednisolone present in an amount of about 5 mg/kg and the antiepileptic is trimethadione present in an amount of about 200 mg/kg.

In another embodiment, the corticosteroid is methylprednisolone which is present in an amount ranging from about 3 to about 8 mg/kg and the antiepileptic is ethosuximide which is present in an amount ranging from about 180 to about 220 mg/kg. In one preferred embodiment, the corticosteroid is methylprednisolone which is present in an amount of about 5 mg/kg and the antiepileptic is ethosuximide present in an amount of about 200 mg/kg.

In still another embodiment, the corticosteroid is methylprednisolone present in an amount ranging from about 3 to about 10 mg/kg and the antiepileptic zonisamide and is present in an amount ranging from about 40 to about 80 mg/kg. In one preferred embodiment, the corticosteroid is methylprednisolone present in an amount of about 8 mg/kg and the antiepileptic is zonisamide which is present in an amount of about 60 mg/kg.

In some embodiments, the ratio of corticosteroid to antiepileptic may range from about 1:0.1 to 1:100, including ratios of about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100 and at ratios between any of the listed values. In some embodiments, the corticosterioid is methylprednisolone and the antiepileptic is chosen from trimethadione and ethosuximide and the ratio of methylprednisolone to the antiepileptic ranges from about 1:40 to 1:60. More preferably, when the corticosterioid is methylprednisolone and the antiepileptic is chosen from trimethadione and ethosuximide the ratio of methylprednisolone to the antiepileptic is about 1:50. In alternative embodiments, the corticosterioid is methylprednisolone and the antiepileptic is zonisamide and the ratio of methylprednisolone to zonisamide ranges from about 1:4 to 1:12. More preferably, the ratio of methylprednisolone to zonisamide is about 7.5.

In some aspects, the combinations are synergistic. The term “synergistic” refers to an effect in which two or more agents work in synergy to produce an effect that is more than additive of the effects of each agent independently. One measure of synergism can be shown by the Chou-Talalay Combination Index Method. The Chou-Talalay Index method is based on the median-effect equation, and derived from the mass-action law principle, which is the theory that links single entity and multiple entities, and first order and higher order dynamics, encompassing the Michaelis-Menten, Hill, Henderson-Hasselbalch, and Scatchard equations. The Chou-Talalay Combination Index Method gives a combination index (CI) where an additive effect gives a CI=1, synergism gives a CI<1, and antagonism gives a CI>1. See Ting-Chao Chou, 2008, Preclinical versus clinical drug combination studies. Lukemia & Lymphoma, 49:2059-2080.

### (d) Pharmaceutical Composition

The combinations may be formulated for pharmaceutical delivery to a subject. For example, therapeutics may be in the form of free bases or pharmaceutically acceptable acid addition salts thereof. The term “pharmaceutically-acceptable salts” embraces salts commonly used to form alkali metal salts and to form addition salts of free acids or free bases. The nature of the salt may vary, provided that it is pharmaceutically acceptable. Suitable pharmaceutically acceptable acid addition salts of compounds of use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric, and galacturonic acid. Suitable pharmaceutically acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine), and procaine.

As will be appreciated by the skilled artisan, the combinations may be formulated into pharmaceutical compositions suitable for different types of administration. They may be administered locally or systemically. The combinations may be administered orally, parenterally, by inhalation spray, intrapulmonary, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intraarterial, intraperitoneal, intracochlear, or intrasternal injection, or infusion techniques. The therapeutic agents of the present invention may be administered by daily subcutaneous injection or by implants. The agents may be administered in liquid drops to the ear canal, delivered to the scala tympani chamber of the inner ear, or provided as a diffusible member of a cochlear hearing implant. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols may be used. Mixtures of solvents and wetting agents such as those discussed above are also useful.

Solid dosage forms for oral administration may include solutions, capsules, tablets, pills, powders, and granules. In such solid dosage forms, the compounds are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as can be provided in a dispersion of active compound in hydroxypropylmethyl cellulose. In the case of capsules, tablets, and pills, the dosage forms may also comprise buffering agents such as sodium citrate, or magnesium or calcium carbonate or bicarbonate. Tablets and pills may additionally be prepared with enteric coatings.

For therapeutic purposes, formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

### II. Method for Treating or Preventing Non Age-Related Hearing Impairments

Another aspect of the present invention encompasses a method for treating or preventing a non age-related hearing impairment in a subject in need of such treatment. The method comprises administering a combination comprising a corticosteroid and an antiepileptic drug to a subject in an amount therapeutically effective to treat or prevent non age-related hearing impairment in the subject, wherein the therapeutically effective amount of the corticosteroid comprises a dose from about 1 to about 50 mg/kg of the body weight of the subject, and the therapeutically effective amount of the antiepileptic comprises a dose from about 20 to about 350 mg/kg of the body weight of the subject. The method is applicable for existing non age-related hearing impairments, and for the prevention of non age-related hearing impairments.

### (a) Combinations

The combinations described in section (I) are suitable for the method for treating non age-related hearing impairments.

### (b) Subjects

The method comprises administration to a subject. The subject may be a human. In other embodiments, the subject may be a veterinary subject. Non-limiting examples of suitable veterinary subjects include companion animals such as cats, dogs, rabbits, horses, and rodents such as gerbils; agricultural animals such as cows, cattle, pigs, goats, sheep, horses, deer, chickens and other fowl; zoo animals such as primates, elephants, zebras, large cats, bears, and the like; and research animals such as rabbits, sheep, pigs, dogs, primates, mice, rats and other rodents.

In some aspects, the invention provides a method to treat damage to a sensory hair cell or a cochlear neuron due to any one or more of ototoxic drug exposure, sound trauma, surgical trauma (i.e., related to the surgical removal of a tumor on cranial nerve VIII), physical trauma (i.e., due to a fracture of the temporal bone affecting the inner and middle ear or due to a shearing injury affecting cranial nerve VIII), mercury, lead, toluene, disease, infection, and a genetic disorder. Typically, the severity of damage that may be treated will depend in large part on the nature and extent of an individual's exposure to any of the above-described stressors.

Ototoxic drugs include several types of antibiotics, such as aminoglycosides (i.e., gentamicin, erythromycin, streptomycin, tobramycin, neomycin, amikacin, netilmicin, etc.) and macrolide antibiotics (i.e., clarithromycin, azithromycin, roxithromycin), certain chemotherapeutic agents (i.e., actinomycin, bleomycin, cisplatin, carboplatin, nitrogen mustard, vincristine, dichloromethotrexate), certain diuretics, (i.e., furosemide (Lasix), bumetanide (Bumex), ethacrynic acid (Edecrin)), and NSAIDs as well as certain analgesics (i.e., Advil® and Motrin® (Ibuprofen), Aleve®, Naprosyn, Anaprox (Naproxen), Feldene, Dolobid, Indocin, Lodine, Relafin, Toradol, Volteran, Salicylates (aspirin, disalcid, Bufferin®, Ecotrin®, Trilisate, Ascriptin, Empirin, Excedrin®, Fiorinal).

In a further embodiment, the therapeutic agents are administered to the subject to treat or prevent sound trauma. Sound trauma is a common source of hearing loss. In general, sound is characterized by its intensity (experienced as loudness) and frequency (experienced as pitch), and it is the intensity and duration of a noise exposure that determines the potential for harm to hair cells and cochlear neurons. Sound intensity is measured as sound pressure level (SPL) in a logarithmic decibel (dB) scale. The present invention may be utilized to treat sound having a variety of SPL and dB levels. Noise exposure is commonly measured in units of dB(A), a unit based on a scale weighted toward higher frequency sounds, to which the human ear is more sensitive. In certain embodiments, chronic sound exposure may be treated. Chronic exposures equal to an average SPL of 85 dB(A) or higher for an eight-hour period can cause permanent hearing loss. By way of example, a conversation exposes an individual to an SPL of 60 dB(A); a lawnmower exposes an individual to an SPL of 90 dB(A); and, stereo headphones expose an individual to an SPL of 110-120 dB(A). For more information regarding the SPLs of common types of noises and the risks of various noise exposures, see Noise-Induced Hearing Loss, by Peter M. Rabinowitz, M.D., M.P.H. (American Family Physician, May, 2000), which is hereby incorporated by reference. Examples of some common noise exposures include industrial/work related noise, i.e., jet takeoff, locomotive noise, recreational (non-work related) noise, gun shot noise, noise from chain saws and other power tools, amplified music, noise from recreational vehicles, such as snowmobiles, water craft, and motorcycles, and noise from some types of children's toys. Industrial/work related noise typically causes a “noise notch,” with hearing loss occurring at mid-high frequencies bilaterally. Firearms, which are owned by some sixty million Americans, and other unilateral sources of noise cause more circumscribed lesions. The methods of the present invention can be used to treat damage to a sensory hair cell or a cochlear neuron due to any of the above-discussed noise exposures or any other noise exposure that has the potential to cause damage to the hair cell; the methods can be used before, during, and/or after the noise exposure.

In a further embodiment, the therapeutic agent may be administered to a subject to treat or prevent hearing loss associated with a disease. Generally speaking, diseases associated with hearing loss include most notably, Meniere's disease. Meniere's disease is associated with several symptoms, and not all sufferers exhibit the same symptoms. The four symptoms most commonly associated with Meniere's disease are vertigo or dizziness, fluctuating hearing loss, tinnitus, sensation of pressure in one or both ears. Meniere's disease frequently begins with one symptom, gradually progressing to include other symptoms; a diagnosis may be made in the absence of all four classic symptoms. Hearing loss associated with Meniere's disease may be unilateral (in one ear) or bilateral (in both ears) and commonly involves lower frequency sounds. Hearing loss may become progressively worse and may become permanent. Some individuals with unilateral hearing loss (by some accounts, as many as 50%) will develop bilateral hearing loss. Some individuals lose hearing entirely in one or both ears. Tinnitus may also worsen over time. The methods of the present invention may be used to treat or prevent damage due to Meniere's disease, at several stages of the disease, including before symptoms of hearing loss appear and after one or more symptoms of the disease have subsided. The methods of the present invention may also be used to treat those at risk for developing Meniere's disease. In addition, the methods of the present invention may be used to treat tinnitus.

Tinnitus, the perception of sound in the absence of acoustic stimulus (i.e., ringing, roaring, chirping, whooshing), is a stressful and sometimes incapacitating condition. The perceived sound may be intermittent or constant and its volume may vary from a quiet sound to a sound that drowns out all other sounds. Tinnitus may be objective, i.e., the sound can be detected by a physician, or subjective, i.e., the sound is only detected by the patient. Subjective tinnitus is most common. There is no cure for tinnitus; treatment usually involves treating the underlying cause of the tinnitus, i.e., Meniere's disease, head injury, stress, depression, or teaching patients coping techniques. For example, an individual suffering from chronic tinnitus may develop ways of masking the tinnitus sound with an artificial sound, i.e., from an electronic device. Tinnitus Retraining Therapy, which includes a combination of masking and psychological counseling, is commonly used with tinnitus patients. It is believed that there are two different types of subjective tinnitus, somatic tinnitus, which is linked to disorders within the head or neck but outside the ear, and otic tinnitus, which is linked to inner ear disorders, including disorders of the acoustic nerve. Other types of tinnitus include external ear tinnitus (which may involve the external ear canal or the ear drum), middle ear tinnitus (which may involve the middle ear chamber or the eustachian tube), inner ear tinnitus (which may involve the hair cells of the inner ear), nerve pathway tinnitus (which may involve cranial nerve VIII), and brain tinnitus (which may involve swelling of the brain). The methods of the present invention may be used to treat or prevent any type or degree of tinnitus, including subjective, objective, somatic, otic, external ear, middle ear, inner ear, nerve pathway, and brain tinnitus. The methods of the present invention may also be combined with any existent treatment for tinnitus, such as Tinnitus Retraining Therapy.

In an additional embodiment, the therapeutic agent is administered to treat or prevent hearing loss resulting from an infection. Several types of infections are associated with hearing loss, including bacterial and viral infections. Examples of such infections include labyrinthitis, syphilis, meningitis, mumps, and measles. Labyrinthitis refers to the inflammation of the inner ear or the nerves connecting the inner ear to the brain. Inflammation of the cochlea results in tinnitus and/or hearing loss. This inflammation may be the result of a bacterial or a viral infection. With regard to bacterial infections, bacteria and/or bacterial toxins may enter the inner ear as a result of bacterial meningitis or due to a rupture in the membranes that separate the middle ear from the inner ear (i.e., due to otitis media or perilymph fistula—a leakage of inner ear fluid to the middle ear associated with head trauma, physical exertion, or barotraumas). Viruses that cause inflammation in the inner ear are believed to enter the inner ear through the blood stream, e.g., via a local or systemic infection. Examples of common viruses that have been associated with labyrinthitis include influenza, measles (rubeola), mumps, German measles (rubella), herpes, hepatitis, polio, and Epstein-Barr. The methods of the present invention may be used to treat damage due to an infection, at any stage of the infection, including before symptoms of hearing loss appear and after one or more symptoms of the infection have subsided. The methods of the present invention may also be used to treat those at risk for acquiring an infection associated with hearing loss.

Hair cells and cochlear neurons may also be damaged or malfunction as a result of an underlying genetic disorder. By way of example, several genes have been identified as encoding key proteins associated with the stereocilia of hair cells, namely myosins VI, VIIA, and XV. Mutations in these genes impair transduction and have been shown to lead to deafness. A mutation in the murine gene encoding myosin VI results in progressive fusion of stereocilia; a mutation in the murine myosin XV gene results in short stereocilia; and a mutation in the murine myosin VIIA gene results in progressive disorganization of the stereocilia bundle. Mutations in myosins VIIA and XV have been associated with human deafness, also. Mutations in proteins that interact with one of these three myosins may also result in hearing impairment. For example, the protein harmonin, a protein present in stereocilia and known to underlie Usher syndrome type 1C (discussed in more detail below), may interact with myosin VIIA. The transmembrane protein vezatin, which binds to the myosin VIIA tail, is believed to be involved in stereocilia organization. Mutations in either one of these interacting proteins may be associated with hearing impairment. Additionally, mutated cadherin-related genes have been associated with the deaf mouse mutants waltzer and Ames waltzer, both of which show evidence of disorganization of the stereocilia bundle; the products of these genes may be involved in linking adjacent stereocilia. A frame shift mutation in the espin gene, which encodes the essential cytoskeletal component of stereocilia espin, has been associated with the deaf mouse mutant jerker. Other examples of genetic mutations associated with malfunctioning hair cells and resultant hearing impairment include mutations in the Atp2b2 gene and in the otoferlin gene (OTOF). The Atp2b2 gene is believed to encode a calcium pump in hair cells and is associated with the deaf waddler mouse mutant, a mutant that lacks a calcium pump. Mutations in the human OTOF gene have been reported in some cases of dominantly inherited, progressive deafness. Any of the above-described mutations may be detected using any one of a number of genetic testing methods known in the art. The methods of the present invention may be used to treat orprevent hearing loss resulting from any of above-described genetic disorders.

The invention also provides methods that may be used to treat or prevent hearing loss resulting from damage to a sensory hair cell or a cochlear neuron due to a combination of factors, such as a genetic disorder, ototoxic drug exposure, sound trauma, surgical trauma, physical trauma, mercury, lead, toluene, disease, and infection. For example, sound trauma is often a co-factor in hearing loss due to ototoxic drug exposure. Thus, those who suffer from hearing impairments due to an ototoxic exposure may be at much greater risk for further hearing impairments due to sound trauma. Those who consume salt in large quantities may also be more vulnerable to sound trauma.

In certain further aspects, the invention provides methods that may be used to treat or prevent non age-related hearing impairments in a subject in need of such treatment. Particularly, in some aspects, the invention provides a method to treat or prevent non age-related hearing impairments due to any one or more of ototoxic drug exposure, sound trauma, surgical trauma (i.e., related to the surgical removal of a tumor on cranial nerve VIII), physical trauma (i.e., due to a fracture of the temporal bone affecting the inner and middle ear or due to a shearing injury affecting cranial nerve VIII), mercury, lead, toluene, disease, infection, and a genetic disorder. Typically, the severity of damage that may be treated or prevented will depend in large part on the nature and extent of an individual's exposure to any of the above-described stressors. The above discussion of these stressors, i.e., types of ototoxic drugs, sound traumas, physical traumas, in the context of treating damage to sensory hair cells and cochlear neurons, is applicable in the context of treating or preventing non age-related hearing impairments, as well. As with methods of treating or preventing hearing loss resulting from damage to hair cells and cochlear neurons, methods of treating non age-related hearing impairments associated with any of the above stressor exposures include administering the therapeutic agents of the invention, prior to, during, or after the stressor exposure, or to individuals at risk for the stressor exposure. Additionally, the invention provides methods of treating non age-related hearing impairments due to a genetic disorder, including autosomal dominant, autosomal recessive, or X-linked disorders. Examples of genetic disorders in which hearing impairments may be a symptom are Down syndrome (abnormality on a gene), Usher syndrome (type 1, 2, and 3) (autosomal recessive), Treacher Collins syndrome (autosomal dominant), Fetal alcohol syndrome (genetic abnormality), Crouzon syndrome (autosomal dominant), Alport syndrome (X-linked), Stickler syndrome (autosomal dominant), Waardenburg Syndrome, Pendred Syndrome, Norrie Syndrome, Branchial-oto-renal syndrome, and Jervell and Lange-Nielsen syndrome. The methods of the invention may be used to treat or prevent non age-related hearing impairments resulting from any of the above genetic disorders.

### (c) Administration

Administration of the combinations may occur in various formulations and through various routes of administration including parenteral, oral, by injection, by inhalation spray, or rectally. Preferred methods of administration are oral administration and intravenous administration. In an exemplary embodiment, a combination of the invention is administered intravenously. In another exemplary embodiment, a combination of the invention is administered orally.

The combinations may be administered in a variety of intervals. In some embodiments, the combinations are administered one or more times daily. In other embodiments, the combinations may be administered prior to an event leading to non age-related hearing impairments such as exposure to loud sounds. In such embodiments, the combinations may be administered from 2 hours to 2 days prior to the exposure. In various embodiments, the combinations may be administered about 1, 2, 3, 4, 5, 6, 7, 8, 9, 10, 11, 12, 14, 16, 18, 20, 22, 24, 26, 28, 30, 32, 34, 36, 38, 40, 42, 44, 46, or 48 hours prior to exposure.

In still another embodiment, administration may occur after exposure to an event leading to non age-related hearing impairments. In some embodiments the combinations may be administered up to 2 days after the exposure. In various embodiments, the combinations may be administered after exposure, 15 minutes post-exposure, 30 minutes post-exposure, 1 hour post-exposure, 2 hours post-exposure, 3 hours post-exposure, 4 hours post-exposure, 5 hours post-exposure, 6 hours post-exposure, 7 hours post-exposure, 8 hours post-exposure, 9 hours post-exposure, 10 hours post-exposure, 11 hours post-exposure, 12 hours-post exposure, 14 hours post-exposure, 16 hours post-exposure, 18 hours post-exposure, 20 hours post-exposure, 22 hours post-exposure, 24 hours post-exposure, 26 hours post-exposure, 27 hours post-exposure, 28 hours post-exposure, 30 hours post-exposure, or more.

### DEFINITIONS

As used herein, the term “effective amount,” refers to the amount of corticosteroid or antiepileptic required to achieve an intended purpose for both prevention and treatment.

The term “hearing impairment” refers to a defect in the ability to perceive sound and includes partial hearing loss, complete hearing loss, deafness (complete or partial), and tinnitus, the perception of non-existent sounds, i.e., a buzzing in the ear. The hearing impairment may be due to noise, surgical procedures, toxins, or other pathological conditions. Hearing impairment includes sensorineural hearing loss, conductive hearing loss, combination hearing loss, mild (between 25 and 40 dB), moderate (between 41 and 55 dB), moderately severe (between 56 and 70 dB), severe (between 71 and 90 dB), and profound (90 dB or greater) hearing loss, congenital hearing loss, pre-lingual and post-lingual hearing loss, unilateral (affecting one ear) and bilateral (affecting both ears) hearing loss, or any combination of these, i.e., sensorineural/severe/postlingual/bilateral.

The term “treat” or “treatment” as used herein in the context of hearing loss, loss of sense of balance, death of sensory hair cells or cochlear neurons, sensorineural hearing loss, or damage to sensory hair cells or cochlear neurons and the like, includes preventing the damage before it occurs, or reducing loss or damage after it occurs.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction for Examples 1-6

Noise-induced hearing loss (NIHL) is the single predominant health hazard posed by occupational and recreational settings. Although promising approaches have been identified for reducing NIHL mainly based on the free radical pathway, currently no effective pharmacological agents are approved by the FDA to diminish permanent hearing loss. Development of an efficacious treatment has been hampered by the complex array of cellular and molecular pathways involved in NIHL.

One major mechanism underlying NIHL is the increase of mitochondrial free radical formation due to noise-induced intense metabolic activity in the cochlea. The involvement of this pathway in NIHL is strongly supported mainly by three lines of evidence: (1) a noise-induced increase of free radicals is observed in stria vascularis, outer hair cells (OHCs), supporting cells of the organ of Corti, and spiral ganglion, and this free radical insult can continue up to 14 days post-exposure; (2) the depletion of endogenous antioxidants and reduction of superoxide dismutase results in increased susceptibility to NIHL; (3) an enhancement of antioxidants attenuates NIHL. Thus, it is not surprising that attempts to prevent NIHL by antioxidant agents have become the focus of much research. Because most of these interventions with single chemicals are only partially effective in preventing NIHL, a few studies have started to intervene at multiple sites in the free radical pathway or in combinations of other pathways with a synergic effect observed in some but not all studies. These studies provide compelling evidence for the role of free radicals in NIHL, but they also suggest that other signaling mechanisms may contribute to NIHL.

Among other main mechanisms contributing to NIHL such as the excitotoxic glutamate at the initial phase or cell death pathways at the end phase of NIHL, two new pathways have emerged: calcium and glucocorticoid (GC) signaling pathways. Disturbance in calcium homeostasis has been suspected to contribute to trauma-induced neuronal injury. Calcium homeostasis in the cochlea can be regulated by several types of calcium channels, which include voltage-gated calcium channels (VGCCs). VGCCs can be divided into two groups: high-voltage activated and low-voltage activated calcium channels. Blockers of L-type calcium channels (high-voltage activated channels) were reported to attenuate NIHL. However, other studies have not supported any protective effect of these blockers. The inventors have found that NIHL may be prevented by the administration of anticonvulsant drugs blocking T-type calcium channels either before or after the noise exposure. Inhibition of T-type calcium channels also protects neurons after stroke. Thus, it is possible that pharmacological modulation of T-type calcium channels may prevent injury-induced alterations of calcium homeostasis, which may contribute to NIHL.

Another major molecular mechanism involved in NIHL is the GC signaling pathway. Synthetic GCs are already used clinically to treat hearing loss in a variety of cochlear disorders such as autoimmune inner ear disease, tinnitus and Meniére's disease. In addition, extensive evidence suggests an important role of GC pathways in NIHL. First, stressful preconditioning such as restraint, heat exposure, or even low-level sound in animal models has been found to be protective against NIHL. Second, because the noise exposure itself is a stressful event, a pretreatment of blockers for GC signaling make animals more susceptible to NIHL. Third, synthetic GCs such as dexamethasone and methylprednisolone can protect against NIHL. Fourth, although GCs can bind to both GC receptors (GR) and minerocorticoid receptors, antagonists against minerocorticoid receptors have no effect on NIHL. Finally, a series of studies have systematically revealed the role of GR signaling pathways in NIHL.

Similar to the NIHL intervention methods based on the free radical pathway, current interventions based on synthetic GC drugs or anticonvulsants blocking T-type calcium channels show limited success in preventing NIHL. However, given these two current interventions are from two completely different drug families, which most likely act on different molecular pathways underlying NIHL, the identification of specific drug combinations from these two drug families that may act in synergistic ways against NIHL is a logical next step.

### Example 1

### Development of a Combination Therapy for Treating or Preventing Noise-Induced Hearing Loss

### Animals and Drug Treatments

(a) Animals.

C57BL/6J mouse line was purchased from Jackson Laboratories. The new stocks were purchased every two years to avoid possible derivations of substrains. Five mice were housed per cage with food and water available in a noise-controlled environment on a 12-hr light/dark cycle with light onset at 6:00 a.m.

(b) Drug Treatments.

Mice were randomly assigned to either treated or untreated groups. The treatment drugs were injected (i.p.). The control groups were injected with normal saline.

### NIHL Model

Similar to approaches described previously (Shen et al., 2007; Hear Res. 226:52-60), noise exposures were performed in a foam-lined, double-walled soundproof room (Industrial Acoustics). The noise exposure apparatus consisted of a 21×21×11 cm wire cage mounted on a pedestal inserted into a B&K 3921 turntable. The cage was rotated at 1 revolution/80 s within a 42×42 cam metal bar frame. A Motorola KSN1020A piezo ceramic speaker (four total) was attached to each side of the frame. Opposing speakers were oriented not concentrically, but parallel to the cage and driven by independent channels of a Crown D150A power amplifier. Noise was generated by two General Radio 1310 generators and bandpassed at 4.0-45.0 kHz by Krohn-Hite 3550 filters. The overall noise level was measured at the center of the cage using a B&K 4135 ¼ inch microphone in a combination with a B&K 2231 sound level meter set a broadband (0.2 Hz-70 kHz). Mice were exposed in pairs to white noise at 110 dB SPL for 30 min.

### ABR Functional Assay

The mouse cochlea typically responds to frequencies ranging from 2-100 kHz. The most sensitive region of the audiogram is roughly 5-40 kHz. To cover this range, tests were conducted at 10, 20, 30, 40, and 50 kHz. The “near field” sound stimulation and calibration were used in which the speaker is near the ear (7 cm) within the range where the sound field is approximately homogeneous within an imaginary cylinder surrounding the ear. To make sure sound stimuli were constant from animal to animal, a B&K 4135 ¼ inch microphone was placed where the mouse ear would normally be and calibrated before the experiment. Prior to testing, all mice were anesthetized with pentobarbital (60 mg/kg, i.p.) and given atropine sulfate (0.5 mg/kg, i.p.) to reduce respiratory distress. Otoscopic examinations were performed to ensure that tympanic membranes are normal. Core temperature was maintained at 37+/−1° C. using a thermostatically-controlled heating pad in conjunction with a rectal probe (Yellow Springs Instruments Model 73A). Platinum needle electrodes (Grass) were inserted subcutaneously just behind the right ear (active), and at the vertex (reference), and in the back (ground). Electrodes were led to a Grass P15 differential amplifier (100-10,000 Hz, ×100), then to a custom amplifier providing another ×1,000 gain, finally digitized at 30 kHz using a Cambridge Electronic Design Micro1401 in conjunction with SIGNAL™ and custom signal averaging software operating on a 120 MHz Pentium PC. Sinewave stimuli generated by a Wavetek Model 148 oscillator were shaped by a custom electronic switch to 5 ms total duration, including 1 ms rise/fall times. The stimulus was amplified by a Crown D150A power amplifier and output to a KSN1020A piezo ceramic speaker. Toneburst stimuli at each frequency and level were presented 1,000 times at 20/sec. The minimum sound pressure levels required for a response (short-latency negative wave) were determined at selected frequencies, using a 5 dB minimum step size.

### Results

NIHL prevention by the synthetic corticosteroid drugs methylprednisolone and dexamethasone was tested (FIG. 1). ABR thresholds among the control and different dosages of the corticosteroid drugs are shown.

NIHL prevention by the antiepileptic drugs ethosuximide and zonisamide was tested. ABR thresholds among the control and different dosages of the antiepileptic drugs are shown (FIG. 2).

A synergistic effect between the synthetic corticosteroid drug methylprednisolone and the antiepileptic drug zonisamide was shown (FIG. 3). ABR thresholds were about 10 dB lower across four frequencies between the control and treated mice. The methylprednisolone and zonisamide combination was synergistic because of a CI<1.

NIHL treatment using corticosteroid and antiepileptic drugs was also tested using different doses of the drugs. The synergistic effect between the synthetic corticosteroid drug methylprednisolone and the antiepileptic drugs ethosuximide for NIHL treatment was also shown (FIG. 4).

NIHL can also be significantly reduced by a combination of methylprednisolone (5 mg/kg) and the anticonvulsant trimethadione (200 mg/kg) given 24 hours after exposure to the 8-16 kHz OBN at 108 dB SPL for 2 hours in 2 month-old B6.CAST mice (FIG. 5). Permanent hearing loss (more than 20 dB at 10 and 20 kHz) was dramatically reduced by this combination treatment 12 hours post-exposure.

In summary, the median effective dose (ED₅₀) to prevent noise-induced hearing loss was determined for drugs from corticosteroid and antiepileptic drug families. The ED₅₀s were: methylprednisolone (525 mg/kg), dexamethasone (39.4 mg/kg), and zonisamide (125 mg/kg); but ethosuximide had no ED₅₀. In addition, the median effective dose to treat noise-induced hearing loss for all four drugs was determined: methylprednisolone (95.6 mg/kg), dexamethasone (96.3 mg/kg), and zonisamide (2543 mg/kg). The ethosuximide ED₅₀ was determined to be 243 mg/kg.

Significantly, a synergic effect to prevent noise-induced hearing loss by methylprednisolone and zonisamide (CI=0.97) was discovered. There was little evidence for possible synergic effects to treat noise-induced hearing loss by i.p. injections with these two families of drugs, however, by oral administration for two weeks, a significant effect was observed by the two-drug treatment.

### Experimental Methods for Examples 2-6

### Animals

All animal procedures were approved by the Animal Studies Committee at Washington University in St. Louis. The study included a total of 270 C57BL/6J mice aged two months (136 males and 134 females), purchased from The Jackson Laboratory (Bar Harbor, Me., USA). All mice were housed three to five per cage in a noise-controlled environment on a 12 hr light/dark cycle with light onset at 6:00 a.m.

### Drug Application

Animals were subject to one of two protocols, a ‘prevention’ protocol under which drugs were administered two hours prior to a single noise exposure, and a ‘treatment’ protocol wherein drugs were administered 24 hours after noise. All chemicals were obtained from Sigma Chemical Co. (St. Louis, Mo.). Each chemical was dissolved in either physiological saline solution (ethosuximide and zonisamide) or vegetable oil (dexamethasone and methylprednisolone), and then administered intraperitoneally. The control groups were injected with physiological saline solution or vegetable oil. The groups with drug combinations were received two injections (one for each drug).

### Noise Exposure

Noise exposures were performed as described previously (e.g., Bao et al., 2004, Nat. Neurosci. 7:1250-1258), in a foam-lined, double-walled soundproof room (Industrial Acoustics). The noise exposure apparatus consisted of a 21×21×11 cm wire cage mounted on a pedestal inserted into turntable. The cage was rotated at 1 revolution/80 s. A Motorola KSN1020A piezo ceramic speaker (four totals) was attached to each side of a metal frame surrounding the cage. Opposing speakers were driven by independent channels of a Crown D150A power amplifier. Noise was generated by two General Radio 1310 generators and filtered to 4.0-45.0 kHz by Krohn-Hite 3550 filters. The overall noise level was measured at the center of the cage using a B&K 4135 ¼ inch microphone in a combination with a B&K 2231 sound level meter set to broadband (0.2 Hz-70 kHz). Mice were exposed in pairs to white noise at 110 dB sound pressure level (SPL) for 30 min.

### Auditory Brainstem Recording (ABR)

ABR testing was performed prior to treatment, then two weeks after the noise exposure to estimate PTS. ABR thresholds were obtained as described previously (Ohlemiller et al., 2000, Hear. Res. 149:239-247; Bao et al., 2004, J. Neurosci. 25:3041-3045). Prior to testing, all mice were anesthetized with 80 mg/kg ketamine and 15 mg/kg xylazine (i.p.). Otoscopic examination was performed to ensure that tympanic membranes were normal. Core temperature was maintained at 37.5±1.0° C. using a thermostatically-controlled heating pad in conjunction with a rectal probe (Yellow Springs Instruments Model 73A). Platinum needle electrodes (Grass) were inserted subcutaneously just behind the right ear, at the vertex, and in the back (ground). Electrodes were led to a Grass P15 differential amplifier (100-10,000 Hz, x100), then to a custom amplifier providing another ×1,000 gain, and digitized at 30 kHz using a Cambridge Electronic Design Micro1401 in conjunction with SIGNAL™ and custom signal averaging software operating on a 120 MHz Pentium PC. Sinewave stimuli generated by a HP 3445 oscillator were shaped by a custom electronic switch to 5 ms total duration, including 1 ms rise/fall times. The stimulus was amplified by a Crown D150A amplifier and output to a KSN1020A piezo ceramic speaker. Toneburst stimuli at each frequency and level were presented 1,000 times at 20/sec. The minimum sound pressure level required for visual detection of wave I was determined at each frequency using a 5 dB minimum step size. To calibrate sound stimuli, a B&K 4135 ¼ inch microphone was placed where the ear would normally be located.

### Data Analysis

All results were presented as the mean+/−S.D. For the ABR threshold shift data, we used a two-way mixed model analysis of variance (ANOVA) taking consideration of drug concentration, gender, and frequency. To address whether there was a synergy between two drugs, data was applied through the CompuSyn software (ComboSyn, Inc.), which was based on the multi-drug effect equation of Chou-Talalay (Chou, 2006, Pharmacol. Rev. 58:621-681). The median effective dose (ED₅₀) for each drug and the combination index (CI) for each drug pair could be derived based on the input data from this software. The CI<1, CI=1, or CI>1 indicated synergism, additivity, or antagonism of the two-drug combinations, respectively.

### Example 2

### Pre-Exposure Application of Anticonvulsant Drugs

Different dosages of ethosuximide (0, 60, 90, 130, 190, 260 mg/kg) were used to determine its ED₅₀ to prevent NIHL. ABR threshold shifts two weeks after the noise exposure were determined for five frequencies (FIG. 6A). The solid line represents 2-month-old C57BL/J mice receiving physiological saline injection (i.p.). Using a two-way ANOVA with the probability of a type I error set at 0.05, possible differences in drug concentrations or gender were measured. Frequency effects were not studied due to the white-noise exposure used and also the variable nature of ABR thresholds among different frequencies. This ANOVA analysis indicated that there was statistically significant main effect of drug concentrations (F=24.85, df=5, p<2⁻¹⁶), but not of genders (F=0.21, df=1, p=0.65). However, ethosuximide dosage-response patterns were complicated. Post-hoc pair wise comparisons were made using the Tukey/Kramer test. At 60 mg/kg, this drug significantly enhanced NIHL (p<0.002); while at 90 mg/kg, this drug significantly protected against NIHL (p<0.001). However, in the next three higher dosages (from 130 to 260 mg/kg), this protection effect was reduced to no significance (at 260 mg/kg, p=0.80). Due to its nonlinear dosage-responses, ED₅₀s of ethosuximide against NIHL could not be obtained using the Chou-Talalay equation. On the other hand, zonisamide showed a clear dosage-dependent prevention of NIHL (FIG. 6B). For this drug, there was statistically significant drug effects (F=16.51, df=2, p<1.29⁻⁶), and the gender had no effects (F=3.54, df=1, p=0.06). The ED₅₀ for zonisamide against NIHL was 125 mg/kg when calculated using the CompuSyn software (ComboSyn, Inc.).

### Example 3

### Pre-Exposure Application of Synthetic GCs

Similar to the studies of antiepileptic drugs, a significant dosage-dependent prevention of NIHL was discovered for synthetic GCs (FIG. 7). For methylprednisolone, there was statistically significant drug effects (F=3.04, df=2, p<0.05), and the gender had no effects (F=0.32, df=1, p=0.58). For dexamethasone, there was no statistically significant drug effects (F=2.72, df=2, p=0.07), and the gender had no effects as well (F=0.04, df=1, p=0.85). However, based on the Chou-Talalay equation, a dose-dependent PTS reduction was still present for dexamethasone (FIG. 7B). The ED₅₀ for methylprednisolone was 525 mg/kg, and dexamethasone was 39.4 mg/kg against NIHL, when calculated using the CompuSyn software (ComboSyn, Inc.).

### Example 4

### Synergic Effect Against NIHL by Zonisamide and Synthetic GCs

To study possible synergic effects against NIHL by these the different drug families assayed in Examples 2, and 3, efforts were focused on drug combinations between zonisamide and two GCs because no ED₅₀ was obtained for ethosuximide. Since a main objective of the present examples was to reduce side-effects of these drugs by reducing their dosages, efforts were focused on studying their possible synergies against NIHL from their ED₅s to ED₂₀s. A statistically significant drug effect was observed for the drug pair of zonisamide and methylprednisolone at their ED₁₀s (FIG. 8; p<0.005). A synergy was also found for this drug pair (CI=0.97). No synergy was found for the drug pairs of zonisamide and dexamethasone at their ED₅s (CI=1.19) or their ED₁₀s (CI=3.22).

### Example 5

### Post-Exposure Application of the Same Four Drugs

Next, possible therapeutic effects of these individual drugs against NIHL were examined by administrating each drug 24 hours after the noise exposure. For ethosuximide (FIG. 9A), there were statistically significant drug effects (F=3.11, df=2, p<0.05), and the gender effect was also significant (F=3.97, df=1, p<0.05). For zonisamide (FIG. 9B), there were no statistically significant drug effects (F=1.29, df=2, p=0.28), and the gender had no effects as well (F=2.01, df=1, p=0.16). For methylprednisolone (FIG. 10A), there was statistically significant drug effects (F=8.28, df=2, p<0.001), and the gender had no effects (F=1.43, df=1, p=0.24). For dexamethasone, there were statistically significant drug effects (F=4.02, df=2, p<0.05), and the gender had no effects (F=1.16, df=1, p=0.28). The ED₅₀ for ethosuximide at 243 mg/kg, methylprednisolone at 95.6 mg/kg, and dexamethasone at 96.3 mg/kg against NIHL, when calculated using the CompuSyn software (ComboSyn, Inc.). No synergic effects against NIHL by two-drug combinations were ever observed between ethosuximide and methylprednisolone, or ethosuximde and dexamethasone from their ED₅s to ED₂₀s.

### Discussion for Examples 2-5

Based on previous studies, a combination therapy for NIHL was tested that includes ethosuximide and zonisamide from anticonvulsants and dexamethasone and methylprednisolone from synthetic GC drugs. ED₅₀s for these drugs were determined in most cases. A synergistic effect was observed for the drug pair of methylprednisolone and zonisamide to prevent NIHL. Three major issues raised by this study are discussed below.

### Prophylactic and Therapeutic Functions of Anticonvulsants Blocking T-Type Calcium Channels

Previous work by the inventors demonstrated both prophylactic and therapeutic effects against NIHL from trimethadione and ethosuximide, two drugs from the same anticonvulsant family blocking T-type calcium channels. However, one subsequent study found no NIHL prophylactic functions from two similar blockers for T-type calcium channels, mibefradil and flunarizine. Besides the different mouse strains used and different noise exposure conditions, one major difference was that the inventors had fed drugs to mice in their drinking water for three weeks before noise exposure while intraperitoneal injections (i.p.) were used in the other study. In the current study, an injection method was adopted, and similar prophylactic functions were observed for both ethosuximide and zonisamide. Most importantly, a complicated pharmacodynamic pattern against NIHL was observed for ethosuximide (FIG. 6A), which suggested that different dosage ranges could be one reason to explain observed differences between these two studies.

In previous work by the inventors (Shen et al, 2007, Hear. Res. 226:52-60), significant therapeutic effect against NIHL for trimethadione was observed only in male mice. Similarly, in the current Examples, gender difference was also observed for NIHL therapeutic effects of ethosuximide, although both ethosuximide and zonisamide showed no gender differences in their NIHL prophylactic functions. These data suggest a possible gender effect for this drug family to treat NIHL, and furthermore, suggest possible different molecular mechanisms underlying their prophylactic and therapeutic functions against NIHL.

Currently, molecular mechanisms underlying their prophylactic and therapeutic functions are unknown. Previously, a strong expression of the α1H subunit in SGNs was observed, which showed no enhanced survival in treated animals (Shen et al, 2007, Hear. Res. 226:52-60). Furthermore, preliminary studies by the inventors found similar NIHL prophylactic functions of ethosuximide in mice lacking the α1H subunit (data not shown), suggesting that this subunit was not the molecular target for these drugs against NIHL. On the other hand, hair cells and supporting cells appeared to express both α1G and α1I calcium channel subunits, and an OHC protection was observed in our previous study by trimethadione (Shen et al, 2007, Hear. Res. 226:52-60). Thus, these drugs may act on α1G, α1I, or both against NIHL. However, other molecular mechanisms may be involved. For example, flunarizine, another T-type calcium channel blocker, was previously shown to inhibit cisplatin-induced death of cultured auditory cells. The mechanism, however, was proposed to be inhibition of lipid peroxidation and mitochondrial permeability transition, not blockage of T-type calcium channels.

### Prophylactic and Therapeutic Functions of GCs

The ability of GCs to prevent NIHL has been demonstrated in various animal models. However, GCs are known to cause serious dose-dependent side effects, including psychosis, gastritis, hypertension, insulin resistance, sleep disturbances, and aseptic necrosis of the hip. Therefore, the present Examples focus on identifying low prophylactic and therapeutic effective doses for two main GCs-dexamethasone and methylprednisolone. Previous studies showed dexamethasone had no protective effects against NIHL at 1 mg/kg in rats. In mice, methylprednisolone had protective effects against NIHL from 10 to 100 mg/kg, while its therapeutic effects at 30 mg/kg were present only if it was administrated within three hours after the noise exposure. In the current study, therapeutic effects of methylprednisolone against NIHL at 60 mg/kg were discovered even 24 hours after the noise exposure (FIG. 10A), another strong support for the need of detailed pharmacodynamic studies. Most importantly, a synergy effect of methylprednisolone and zonisamide against NIHL was discovered, which allowed the effective dose of methylprednisolone to be as low as 8 mg/kg. This data also provided strong support of different molecular pathways used by these two different drug families to prevent NIHL. On the other hand, no synergy was found for these two drug families to treat NIHL could suggest overlapping pathways by these drugs, or possible weaknesses of the Chou-Talalay method, further discussed below.

### Consideration of the Chou-Talalay Model

Drug combination therapies are highly successful in treating diseases such as HIV infections. They have advantages over single drug therapies such as achieving sufficient therapeutic effect at a lower dose with few side effects. However, uncovering drug combinations by direct screening without any computational analysis is challenging due to the large number of potential combinations. Pharmacodynamics analysis in this study was based on the Chou-Talalay median-effect equation. This equation is derived from the mass-action law principle, which provides the common link between single and multiple drug-target interactions, and first order and higher order dynamics. The Chou-Talalay equation has in fact the same mathematical form as the Hill function. Besides of its usefulness in dose-response trends of each single drug, the resulting CI from this equation offers quantitative definition for additive effect (CI=1), synergism (CI<1), and antagonism (CI>1) in drug combinations. Thus, this equation was widely used in the discovery of drug combinations. Although the Chou-Talaly equation is independent of the drug's mechanisms of action and does not require knowledge of conventional kinetic constants, its assumption that two drugs are mutually exclusive led to underestimating synergistic effects in partially exclusive cases of two drug combinations. This possible underestimation could be the cause for no synergy found for therapeutic effects of drug combinations between antiepileptic and GC drug families.

What is claimed is:

1. A combination, the combination comprising a glucocorticosteroid and an antiepileptic drug selected from the group consisting of an oxazolidinedione, a sulfonamide, and a succinimide, in an amount therapeutically effective to treat or prevent non age-related hearing impairments in a subject, wherein the therapeutically effective amount of the glucocorticosteroid comprises a dose ranging from about 1 to about 50 mg/kg of the body weight of the subject, and the therapeutically effective amount of the antiepileptic comprises a dose ranging from about 20 to about 350 mg/kg of the body weight of the subject.

2. The combination of claim 1, wherein the glucocorticosteroid is methylprednisolone.

3. The combination of claim 2, wherein the therapeutically effective amount of methylprednisolone comprises a dose ranging from about 3 to about 20 mg/kg of the body weight of the subject.

4. The combination of claim 1, wherein the antiepileptic drug is chosen from ethosuximide, trimethadione, zonisamide, and derivatives thereof.

5. The combination of claim 1, wherein the antiepileptic drug is ethosuximide and the therapeutically effective amount comprises a dose ranging from about 150 to about 250 mg/kg of the body weight of the subject.

6. The combination of claim 1, wherein the antiepileptic drug is trimethadione and the therapeutically effective amount comprises a dose ranging from about 150 to about 250 mg/kg of the body weight of the subject.

7. The combination of claim 1, wherein the antiepileptic drug is zonisamide and the therapeutically effective amount comprises a dose ranging from about 40 to about 80 mg/kg of the body weight of the subject.

8. The combination of claim 1, wherein the glucocorticosteroid is methylprednisolone, and the therapeutically effective amount of methylprednisolone comprises a dose of about 8 mg/kg of the body weight of the subject, and the antiepileptic drug is zonisamide and the therapeutically effective amount of zonisamide comprises a dose of about 60 mg/kg of the body weight of the subject.

9. The combination of claim 1, wherein the glucocorticosteroid is methylprednisolone, and the therapeutically effective amount of methylprednisolone comprises a dose of about 5 mg/kg of the body weight of the subject, and the antiepileptic drug is ethosuximide, and the therapeutically effective amount of ethosuximide comprises a dose of about 200 mg/kg of the body weight of the subject.

10. The combination of claim 1, wherein the glucocorticosteroid is methylprednisolone, and the therapeutically effective amount of methylprednisolone comprises a dose of about 5 mg/kg of the body weight of the subject, and the antiepileptic drug is trimethadione, and the therapeutically effective amount of trimethadione comprises a dose of about 200 mg/kg of the body weight of the subject.

11. A method for treating or preventing a non age-related hearing impairment in a subject in need of such treatment, the method comprising administering a combination comprising a glucocorticosteroid and an antiepileptic drug selected from the group consisting of an oxazolidinedione, a sulfonamide, and a succinimide, to a subject in an amount therapeutically effective to treat a non age-related hearing impairment in the subject, wherein the therapeutically effective amount of the glucocorticosteroid comprises a dose ranging from about 1 to about 50 mg/kg of the body weight of the subject, and the therapeutically effective amount of the antiepileptic comprises a dose ranging from about 20 to about 350 mg/kg of the body weight of the subject.

12. The method of claim 11, wherein the glucocorticosteroid is methylprednisolone.

13. The method of claim 12, wherein the therapeutically effective amount of methylprednisolone comprises a dose ranging from about 3 to about 20 mg/kg of the body weight of the subject.

14. The method of claim 11, wherein the antiepileptic drug is chosen from ethosuximide, trimethadione, zonisamide, and derivatives thereof.

15. The method of claim 11, wherein the antiepileptic drug is trimethadione and the therapeutically effective amount comprises a dose ranging from about 150 to about 250 mg/kg of the body weight of the subject.

16. The method of claim 11, wherein the antiepileptic drug is ethosuximide and the therapeutically effective amount comprises a dose ranging from about 150 to about 250 mg/kg of the body weight of the subject.

17. The method of claim 11, wherein the antiepileptic drug is zonisamide and the therapeutically effective amount comprises a dose ranging from about 40 to about 80 mg/kg of the body weight of the subject.

18. The method of claim 11, wherein the corticosteroid is methylprednisolone, and the therapeutically effective amount of methylprednisolone comprises a dose of about 5 mg/kg of the body weight of the subject, and the antiepileptic drug is ethosuximide, and the therapeutically effective amount of ethosuximide comprises a dose of about 200 mg/kg of the body weight of the subject.

19. The method of claim 11, wherein the corticosteroid is methylprednisolone, and the therapeutically effective amount of methylprednisolone comprises a dose of about 8 mg/kg of the body weight of the subject and the antiepileptic drug is zonisamide, and the therapeutically effective amount of zonisamide comprises a dose of about 60 mg/kg of the body weight of the subject.

20. The method of claim 11, wherein the corticosteroid is methylprednisolone, and the therapeutically effective amount of methylprednisolone comprises a dose of about 5 mg/kg of the body weight of the subject, and the antiepileptic drug is trimethadione, and the therapeutically effective amount of trimethadione comprises a dose of about 200 mg/kg of the body weight of the subject.

21. The method of claim 11, wherein the method comprises oral administration.

22. The method of claim 11, wherein the method comprises administration by injection.
